(19) United States
(12) Patent Application Publication
(10) Pub. No.: US 2016/0278853 A1
(43) Pub. Date: Sep. 29, 2016

(54) A BASKET CATHETER AND METHOD OF MANUFACTURING

(71) Applicant: CATHRX LTD, Homebush Bay, New South Wales (AU)

(72) Inventors: David Ogle, Cowan (AU); Roman Greifeneder, Bexley (AU)

(21) Appl. No.: 15/037,990

(22) PCT Filed: Oct. 15, 2014
(86) PCT No.: PCT/AU2014/000981

§ 371 Date: May 19, 2016

(30) Foreign Application Priority Data
Nov. 29, 2013 (AU) 2013904638

(51) Int. Cl. A61B 18/14; A61B 5/042

(52) U.S. Cl. CPC A61B 18/1492; A61B 5/0422; A61B 2017/00526

(57) ABSTRACT

A catheter sheath for a basket catheter that includes a plurality of electrical leads, each having a proximal end and a distal end and a lumen extending from the proximal end to the distal end. Each electrical lead includes a tubular member of non-conductive material, a plurality of electrical conductors extending from the proximal end to the distal end laid on the non-conductive tubular member, and an outer layer of non-conductive material applied over the electrical conductors to cover the conductors. The catheter sheath further includes one or more electrodes on a distal portion of each electrical lead in electrical communication with at least one of the plurality of electrical conductors through the outer layer. An elongate shape-forming member is received in the lumen of each of the plurality of electrical leads. The shape-forming member imparts an arched shape to the distal portion of each of the electrical leads so as to form a basket shape to a distal portion of the catheter sheath. The plurality of electrical leads are bundled together at their distal ends and proximal the distal arched portion of each electrical lead.

### CROSS-REFERENCE TO RELATED APPLICATIONS

This application is a national phase entry under 35 U.S.C. §371 of International Patent Application PCT/AU2014/000981, filed Oct. 15, 2014, designating the United States of America and published in English as International Patent Publication WO 2015/077816 A1 on Jun. 4, 2015, which claims the benefit under Article 8 of the Patent Cooperation Treaty to Australian Patent Application Serial No. 2013904638, filed Nov. 29, 2013.

### TECHNICAL FIELD

This disclosure relates, generally, to a basket catheter and a method of manufacturing a basket catheter.

### BACKGROUND

Any discussion of the prior art throughout the specification should in no way be considered as an admission that such prior art is widely known or forms part of common general knowledge in the field.

Electrophysiology catheters are commonly used in medical practice to examine and treat the heart. They may be inserted into the cardiovascular system of the patient through small punctures in the skin. They may then extend through a vein into the heart where they sense the electrical activity of the heart. Some of the electrophysiology catheters may be able to treat the heart by ablating the appropriate areas of the heart in case of certain types of aberrant electrical activity.

Several different electrode designs have been developed for treating heart arrhythmias such as atrial flutter and atrial fibrillation. One of such designs is a multi-electrode basket catheter where the distal end of the catheter includes several limbs that expand to a spherical shape to enable three-dimensional mapping of the pulmonary vein or atria. A multi-electrode basket catheter is useful for circumferential mapping and treatment of atrial fibrillation but the prior art basket catheters are somewhat limited in the coverage of the mapping area.

### BRIEF SUMMARY

It is an object of this disclosure to overcome or ameliorate at least one of the disadvantages of the prior art, or to provide a useful alternative.

In an aspect, there is provided a catheter sheath for a basket catheter, which includes: a plurality of electrical leads, each having a proximal end and a distal end and a lumen extending from the proximal end to the distal end, the electrical leads each including a tubular member of non-conductive material, a plurality of electrical conductors extending from the proximal end to the distal end laid on the non-conductive tubular member, and an outer layer of non-conductive material applied over the electrical conductors to cover the conductors. The catheter sheath further includes one or more electrodes on a distal portion of each electrical lead in electrical communication with at least one of the plurality of electrical conductors through the outer layer. An elongate shape-forming member received in the lumen of each of the plurality of electrical leads, the shape-forming member imparting an arched shape to the distal portion of each of the electrical leads so as to form a basket shape to a distal portion of the catheter sheath, the plurality of electrical leads being bundled together at their distal ends and proximal the distal arched portion of each electrical lead. Preferably, the plurality of electrical conductors are helically arranged about the tubular member.

In an embodiment, a sleeve is received on the bundle of electrical leads, the sleeve being axially displaceable between a first, extended position in which each of the plurality of electrical leads is collapsed into a substantially rectilinear shape, and a second, retracted position in which the distal portion of each of the plurality of electrical leads adopts the arched shape imparted by the shape-forming member.

In an embodiment, the electrical leads proximal the distal arched portion of the electrical leads are bundled together by an adhesive, by using tubing molded over the electrical lead or by using a heat shrink. The distal ends of the plurality of electrical leads are preferably connected together by a connector element. The connector element may include a flexible portion for receiving the distal ends of the plurality of electrical leads, the flexible portion allowing the electrical leads to collapse when the catheter sheath is inserted into a sleeve or an introducer.

There is also provided a method of fabricating a catheter sheath for a basket catheter, the method comprising: providing a plurality of electrical leads, each having a proximal end and a distal end, and a lumen extending from the proximal end to the distal end, each electrical lead including a tubular member of non-conductive material, a plurality of electrical conductors extending from the proximal end to the distal end laid on the non-conductive tubular member, and an outer layer of non-conductive material applied over the electrical conductors to cover the conductors. At least one of the plurality of electrical conductors are accessed and an electrode is formed on a distal portion of the electrical lead in electrical connection with the at least one of the electrical conductors. The method further includes inserting a shape-forming member in the lumen of each of the plurality of electrical leads, the shape-forming member imparting an arched shape to the distal portion of each of the electrical leads so as to form a basket shape to a distal portion of the catheter sheath, and bundling the plurality of electrical leads together at their distal ends and proximal the distal arched portion of each electrical lead.

In an embodiment, the method includes helically arranging the plurality of electrical conductors on and about the tubular member.

In an embodiment, the method includes inserting a sleeve over the bundle of electrical leads, the sleeve being axially displaceable between a first, extended position in which each of the plurality of electrical leads is collapsed into a substantially rectilinear shape, and a second, retracted position in which each of the plurality of electrical leads adopts the arched shape imparted by the shape-forming member.

### BRIEF DESCRIPTION OF THE DRAWINGS

Preferred embodiments of the disclosure will now be described, by way of example only, with reference to the accompanying drawings in which:

FIGS. 1A and 1B show a distal end of a basket catheter;

FIG. 2 shows a schematic view of an electrical lead manufactured in accordance with an embodiment of the disclosure; and

FIG. 3 shows a step of the process of manufacturing the catheter sheath in accordance with an embodiment of the disclosure.

### DETAILED DESCRIPTION

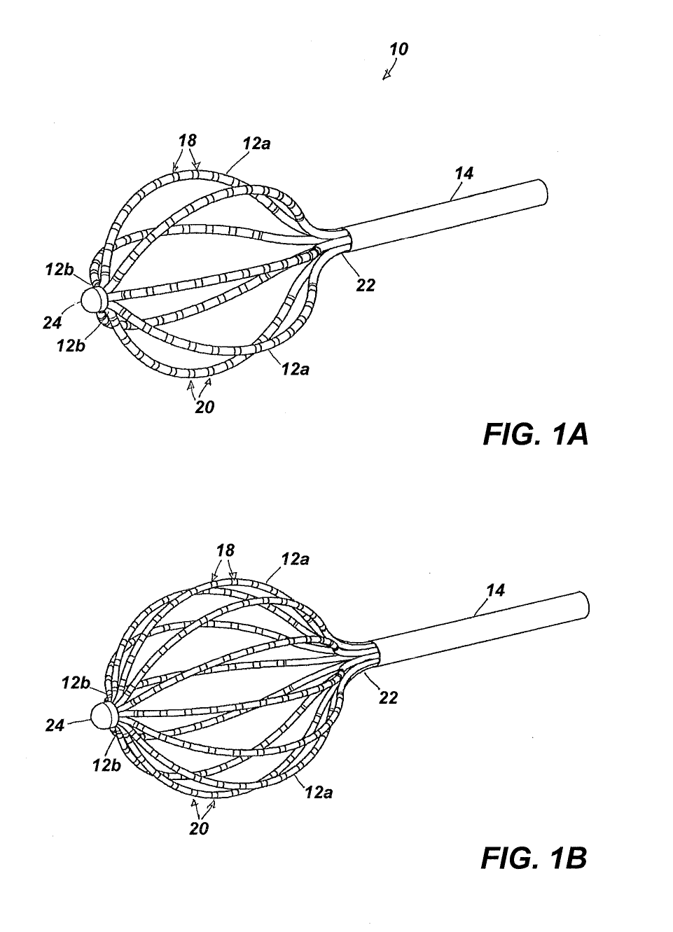

In the drawings, reference numeral 10 generally designates an embodiment of a catheter sheath for a basket catheter made in accordance with a process for manufacturing a basket catheter as described below. The basket catheter 10 is suitable for use as a three-dimensional mapping catheter as well as an ablation catheter for ablating the treatment area. FIGS. 1A and 1B depict the distal end of such a basket catheter. The basket catheter 10 comprises a plurality of elongate electrical leads 12, each lead having one or more electrodes 18, 20 attached to the distal part of the electrical lead. Each electrical lead 12 has a proximal portion (not shown in FIG. 1A or 1B) and a distal portion 12a and a lumen extending from the proximal end to the distal end 12b. A shape-forming member 16 (not shown), such as a shape memory wire made of NITINOL™, is inserted into the lumen of the electrical lead 12. The shape-forming member 16 forms the distal portion 12a of the electrical lead 12 into an arch shape so that each electrical lead forms a spine or an arm at the distal end of the basket catheter. The distal portion 12a of the catheter sheath is formed of the arched arms of the electrical leads 12. The basket formed by distal portions 12a of the electrical leads 12 is substantially spherical in FIGS. 1A and 1B, but it can also be another suitable shape such as an egg shape.

The basket catheter 10 includes an introducer or a sleeve 14 in which the leads 12 of the basket catheter 10 are received. The leads 12 are bundled together by a suitable method such as using an adhesive or by applying heat. Also heat-shrink tubing or a tube molded over the leads may be used to attach the bundle of leads 12 together from a point 22 proximal the distal portion 12a of each electrical lead 12. The introducer includes a steering mechanism (not shown) for steering the basket catheter 10 through the vascular system and the heart of a patient undergoing treatment. It is also possible to use any available introducer that is suitable for catheters. Each arm 12a of the basket catheter 10 consists of an electrical lead 12 that is manufactured in accordance with the method described below. The distal end of each lead is attached to an end connector element 24. The end connector element 24 preferably includes a first flexible element having a tubular receiver for each arm. The distal end 12b of each arm is a snug fit as it slides into the tubular receiver. However, glue or another appropriate adhesive is preferably used to fixedly connect the distal end 12b of each arm 12a to the corresponding tubular receiver. The flexible material of the tubular receivers allows some movement for the arms to enable collapsing of the basket structure to fit it inside the introducer. The first flexible element is covered with a smooth dome or semi-spherical element 24 to allow a smooth unhindered entry into the introducer. Alternatively, distal ends of each shape-forming member are fixedly connected together first by a suitable method such as welding. The flexible electrical leads 12 are then inserted over each shape-forming member and the distal ends 12b of the electrical leads are fixedly attached together by an adhesive or they may also be heat treated or welded to get the leads 12 to attach together. Following the formation of the distal end 12b of the basket catheter 10, the electrical leads 12 are then connected proximal the arms 12a, from point 22 toward the proximal end of the leads 12.

In use, the basket catheter 10 is inserted via the patient's vascular system and the left atrium of the heart into the ostium of the pulmonary vein to be treated where the arrhythmia may be occurring. To facilitate insertion of the basket catheter 10, the bundle of leads are retracted into the introducer 14 so that the arms 12a adopt a collapsed or straight configuration within the introducer 14 as the introducer 14 is steered to the relevant site by an operator. In their collapsed configuration, the arms 12a of the electrical leads 12 lie substantially axial or rectilinear along the axis of the introducer 14. At the treatment site, the introducer 14 is retracted or the arms 12a are urged toward the distal end of the introducer 14 so as to eject the basket out of the distal end of the introducer 14 and allow the shape-forming members to impart the arched shape on the arms 12a.

Sensing of electrical activity at or adjacent the treatment site takes place by sensing electrodes, whereas ablation is effected by ablation electrodes. By using sophisticated computer processing software, it is possible to use an electrode first as a sensing/mapping electrode and then use the same electrode for ablation. The arms 12a may include radio opaque tokens or bands that assist the clinician in placing the basket in the correct treatment area. The radio opaque markers may be arranged at various locations of the arms identifying certain of the electrodes so that the clinician knows exactly where the electrodes are positioned around the treatment site. This is only necessary if the electrodes are not visible under a fluoroscope.

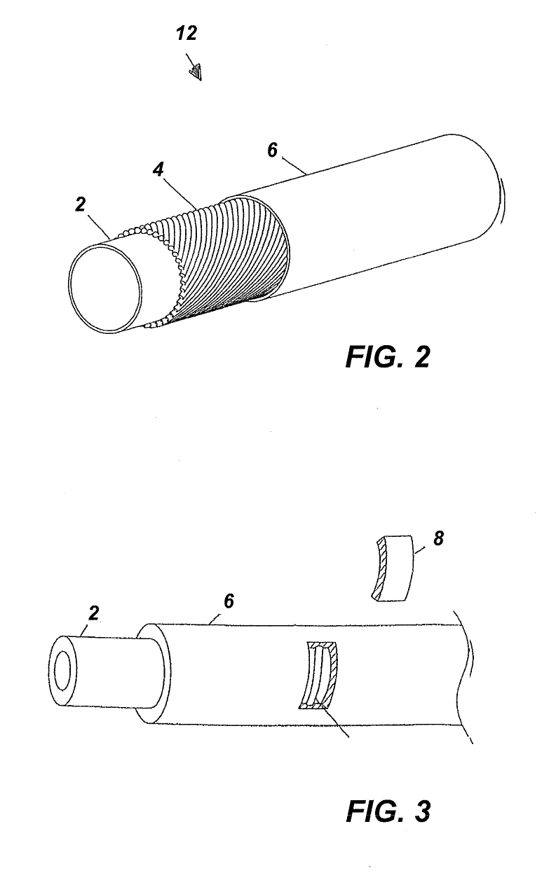

Referring to FIG. 2, the electrical lead 12 has a first inner member 2 made of a non-conductive tubular member. The non-conductive tubular member 2 is formed by extruding a thin layer of a non-conductive material such as polytetrafluoroethylene (PEBAX®, PTFE or TEFLON®) over a mandrel. The tubular member 2 defines a lumen for the shape-forming member of each electrical lead 12.

A plurality of conductors 4 are coiled in a helical manner around the outer surface of the tubular member 2. The conductors 4 are metal wires that are insulated by a polymeric material such as Nylon, polyurethane or a nylon-polyurethane co-polymer. The diameter of the conductor wires is such that the overall electrical resistance is as low as possible. An outer polymeric sleeve 6 is formed, for example, by extrusion, over the conductors 4 to form the electrical lead 12. The outer polymeric sleeve 6 is typically made from materials similar to or the same as the tubular member 2, although the durometer of the materials may vary. The electrical lead 12 comprising the tubular member 2, the conductors 4 and the outer polymeric sleeve 6 is heat treated to secure the outer polymeric sleeve 6 to the tubular member 2, and to the conductors 4. Another method for applying the outer polymeric sleeve 6 over the conductors 4 is to apply molten outer polymeric material over the conductors 4 and allow it to set over time. It will be appreciated that a wall of the electrical lead 12 is, therefore, effectively made up of an inner layer defined by the tubular member 2, the layer made up of the helically wound conductors 4, and an outer layer defined by the outer sleeve 6. The conductors 4 are, in effect, embedded in the wall of the electrical lead and, as there is little, if any, polymeric material between adjacent turns of the conductors 4, there is ability for limited movement between adjacent turns, thereby improving the flexibility of the electrical lead 12. It would also be possible to have the electrical conductors axially laid on the inner tubular member 2 along the tubular member.

One or more electrodes 18, 20 are formed onto the electrical lead by laser cutting a portion 8 (shown in FIG. 3) of the outer polymeric layer 6 to expose a conductor lead and covering the exposed conductor by conductive material such as a platinum ring to form an electrode onto the electrical lead. Laser cutting is accurate and provides a suitable way of removing a portion of the outer polymeric sleeve 6 with ease to produce an opening. If the conductors 4 are insulated, the step of exposing the conductor(s) also cuts and removes the layer of insulation over the wires in addition to cutting and removing a corresponding portion of the outer polymeric sleeve 6.

The opening formed in the outer polymeric sleeve 6 is substantially filled with an electrically conductive paste or adhesive such as a silver-filled epoxy. The electrically conductive adhesive is then overlaid with a conductive bio-compatible material such as a platinum ring that is affixed onto the outside of the electrical lead via a suitable process such as dry swaging, crimping adhesive or a combination thereof. The conductor wire is used for transmission of electrical signals to the handle and the processor or it is used for transmission of ablation energy such as radio frequency (RF) energy to the electrode.

Up to 16 electrodes per arm are achievable because the conductors are embedded in the wall of the electrical lead. Some of the conductors are used for providing an electrical path for the sensing electrodes or the ablation electrodes and some may be used as a thermocouple sensing the temperature of the one or more ablation electrodes. Thermocouple electrodes may thus include two conductor wires attached to the thermocouple electrode. If the thermocouple is associated with an ablating electrode, this electrode may have three conductor wires attached to it. An ablating electrode or a sensing electrode without a thermocouple has only one conductor attached to the electrode. A thermocouple can also be made up by two separate electrodes and, in this case, each electrode would have two conductor wires attached to it, one for sensing or ablating and one for use as part of the thermocouple.

It is an advantage of this basket catheter 10 design that because the conductors are embedded within the wall of the tubular member, each arm can be thinner than prior art basket catheters. This allows for more arms to be inserted into the introducer and thus better coverage of the treatment site is achieved. It is also possible to have more electrodes on each arm, which also allows for better coverage of the treatment area. The Applicant's manufacturing technique for a basket catheter lends itself to the use of up to 16 electrodes per arm and up to 12 arms per catheter. This allows baskets with up to 144 electrodes. As a result, the accuracy of sensing measurements and ablating procedures is improved because the increased number of electrodes allows for a much finer map and greater resolution than has been possible before. The basket catheter 10 assists in plotting a 3-D image of the heart by giving maximum data points to create the 3-D image. Higher electrode counts also allow advanced processing functions used to characterize Complex Fractionated Atrial Electrograms, so called CFAEs, that are currently a target for ablation. It is suggested that having this information available at the processor, may be of value to discriminate where to ablate more precisely.

In addition, a basket catheter 10 is able to be provided that is of substantially smaller diameter than other basket catheters of which the Applicant is aware. Due to the manufacturing techniques employed, the width of the electrical lead 12 may be as little as 3 Fr. The smaller diameter is beneficial for the ease with which the clinician can steer the catheter through a patient's vasculature as the catheter is steered through the patient's body. Furthermore, more electrodes can be carried on each arm without adversely affecting the size of the catheter. Current prior art basket catheters are greatly limited by thicknesses of cables and electrode count per arm.

Reference throughout this specification to “one embodiment,” “some embodiments,” or “an embodiment” means that a particular feature, structure or characteristic described in connection with the embodiment is included in at least one embodiment of this disclosure. Thus, appearances of the phrases “in one embodiment,” “in some embodiments,” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

As used herein, unless otherwise specified, the use of ordinal adjectives “first,” “second,” “third,” etc., to describe a common object, merely indicate that different instances of like objects are referred to, and are not intended to imply that the objects so described must be in a given sequence, either temporally, spatially, in ranking, or in any other manner.

In the claims below and the description herein, any one of the terms “comprising,” “comprised of,” or “which comprises” is an open term that means including at least the elements/features that follow, but not excluding others. Thus, the term “comprising,” when used in the claims, should not be interpreted as being limitative to the means or elements or steps listed thereafter. For example, the scope of the expression “a device comprising A and B” should not be limited to devices consisting only of elements A and B. Any one of the terms “including,” or “which includes,” or “that includes,” as used herein, is also an open term that also means “including at least” the elements/features that follow the term, but not excluding others. Thus, “including” is synonymous with and means “comprising.”

It should be appreciated that in the above description of exemplary embodiments of the disclosure, various features of the disclosure are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment.

Furthermore, while some embodiments described herein include some but not other features included in other embodiments, combination of features of different embodiments are also within the scope of the present disclosure, as would be understood by those skilled in the art. For example, in the following claims, any of the claimed embodiments can be used in any combination.

In the description provided herein, numerous specific details are set forth. However, it is understood that embodiments of the disclosure may be practiced without these specific details. In other instances, well-known methods, structures and techniques have not been shown in detail in order not to obscure an understanding of this description.

Thus, while there has been described what are believed to be the preferred embodiments of the invention, those skilled in the art will recognize that other and further modifications may be made thereto without departing from the spirit of the invention, and it is intended to claim all such changes and modifications as falling within the scope of the invention. For example, any formulas given above are merely representative of procedures that may be used. Functionality may be added or deleted from the block diagrams and operations may be interchanged among functional blocks. Steps may be added or deleted to methods described within the scope of the invention.

It will be appreciated by persons skilled in the art that numerous variations and/or modifications may be made to the disclosure as shown in the specific embodiments without departing from the scope of the disclosure as broadly described. The present embodiments are, therefore, to be considered in all respects as illustrative and not restrictive.

1. A catheter sheath for a basket catheter, the catheter sheath comprising:
a plurality of electrical leads, each having a proximal end and a distal end, and a lumen extending from the proximal end to the distal end, the electrical leads each including a tubular member of non-conductive material, a plurality of electrical conductors extending from the proximal end to the distal end laid on the non-conductive tubular member, and an outer layer of non-conductive material applied over the electrical conductors to cover the conductors;
one or more electrodes on a distal portion of each electrical lead in electrical communication with at least one of the plurality of electrical conductors through the outer layer; and
an elongate shape-forming member received in the lumen of each of the plurality of electrical leads, the shape-forming member imparting an arched shape to the distal portion of each of the electrical leads so as to form a basket shape to a distal portion of the catheter sheath, the plurality of electrical leads being bundled together at their distal ends and proximal the distal arched portion of each electrical lead.

2. The catheter sheath of claim 1, wherein the plurality of electrical conductors are helically arranged about the tubular member.

3. The catheter sheath of claim 2, further comprising a sleeve received on the bundle of electrical leads, the sleeve being axially displaceable between a first, extended position in which each of the plurality of electrical leads is collapsed into a substantially rectilinear shape, and a second, retracted position in which the distal portion of each of the plurality of electrical leads expands and adopts the arched shape imparted by the shape-forming member.

4. The catheter sheath of claim 3, wherein the electrical leads proximal the distal arched portion of the electrical leads are bundled together by an adhesive by using tubing molded over the electrical lead or by using a heat shrink.

5. The catheter sheath of claim 4, wherein the distal ends of the plurality of electrical leads are connected together by a connector element.

6. The catheter sheath of claim 5, wherein the connector element includes a flexible portion for receiving the distal ends of the plurality of electrical leads, the flexible portion allowing the electrical leads to collapse when the catheter sheath is inserted into a sleeve or an introducer and expand when the distal portion of the catheter sheath is urged out of the sleeve.

7. A method of fabricating a catheter sheath for a basket catheter, the method comprising:
providing a plurality of electrical leads each having a proximal end and a distal end, and a lumen extending from the proximal end to the distal end, each electrical lead including a tubular member of non-conductive material, a plurality of electrical conductors extending from the proximal end to the distal end laid on the non-conductive tubular member, and an outer layer of non-conductive material applied over the electrical conductors to cover the conductors;
accessing at least one of the plurality of electrical conductors and forming an electrode on a distal portion of the electrical lead in electrical connection with the at least one of the electrical conductors;
inserting a shape-forming member in the lumen of each of the plurality of electrical leads, the shape-forming member imparting an arched shape to the distal portion of each of the electrical leads so as to form a basket shape to a distal portion of the catheter sheath; and
bundling the plurality of electrical leads together at their distal ends and proximal the distal arched portion of each electrical lead.

8. The method of claim 7, further comprising helically arranging the plurality of electrical conductors on and about the tubular member.

9. The method of claim 8, further comprising inserting a sleeve over the bundle of electrical leads, the sleeve being axially displaceable between a first, extended position in which each of the plurality of electrical leads is collapsed into a substantially rectilinear shape, and a second, retracted position in which each of the plurality of electrical leads adopts the arched shape imparted by the shape-forming member.

10. The method of claim 7, further comprising inserting a sleeve over the bundle of electrical leads, the sleeve being axially displaceable between a first, extended position in which each of the plurality of electrical leads is collapsed into a substantially rectilinear shape, and a second, retracted position in which each of the plurality of electrical leads adopts the arched shape imparted by the shape-forming member.

11. The catheter sheath of claim 1, further comprising a sleeve received on the bundle of electrical leads, the sleeve being axially displaceable between a first, extended position in which each of the plurality of electrical leads is collapsed into a substantially rectilinear shape, and a second, retracted position in which the distal portion of each of the plurality of electrical leads expands and adopts the arched shape imparted by the shape-forming member.

12. The catheter sheath of claim 11, wherein the electrical leads proximal the distal arched portion of the electrical leads are bundled together by an adhesive by using tubing molded over the electrical lead or by using a heat shrink.

13. The catheter sheath of claim 12, wherein the distal ends of the plurality of electrical leads are connected together by a connector element.

14. The catheter sheath of claim 11, wherein the distal ends of the plurality of electrical leads are connected together by a connector element.

15. The catheter sheath of claim 1, wherein the distal ends of the plurality of electrical leads are connected together by a connector element.

16. The catheter sheath of claim 1, wherein the electrical leads proximal the distal arched portion of the electrical leads are bundled together by an adhesive by using tubing molded over the electrical lead or by using a heat shrink.
